(19) United States
(12) Patent Application Publication
(10) Pub. No.: US 2022/0039354 A1
(43) Pub. Date: Feb. 10, 2022

(54) VIRAL SENSOR DETECTION METHODS, METHODS OF TRAINING CANINES FOR VIRAL SENSOR DETECTION AND METHODS OF SCREENING CANINES FOR VIRAL SENSOR DETECTION

(71) Applicant: VIRAL LOGIC LLC, Reston, VA (US)

(72) Inventors: Leigh MARJAMAA-CHOWDHARY, Reston, VA (US); Frank SCHEMBRE, Helotes, TX (US); Michael BARBINE, San Antonio, TX (US); Daniel Warren HAYTER, Sommerset, TX (US); Daniel Glenn HAYTER, Sommerset, TX (US); Paul BUNKER, San Antonio, TX (US)

(21) Appl. No.: 17/394,651

(22) Filed: Aug. 5, 2021

Related U.S. Application Data
(60) Provisional application No. 63/061,259, filed on Aug. 5, 2020.

(51) Int. Cl. A01K 15/02

(52) U.S. Cl. CPC A01K 15/02

(57) ABSTRACT

Provided are methods of detecting a biological pathogen, that include conditioning a canine to search for and passively indicate a specific odor indicating presence of the pathogen; permitting the canine trained to detect the pathogen using canine olfaction, to roam a room or other area to search for the virus; and determining from a passive reaction of the canine, whether the pathogen is present in a location or whether an individual is infected with or carrying the pathogen. The canine is conditioned to search for and passively indicate a specific odor indicating presence of the pathogen using one or more biomarkers selected from the group consisting of sweat, saliva and a pathogen. The sweat may be from human tissue. Also provided are methods of training a canine for biological pathogen detection.

### RELATED APPLICATION

This application claims the benefit of U.S. Provisional Application 63/061,259 filed on Aug. 5, 2020, the contents of which are hereby incorporated herein by reference in its entirety

### FIELD

The present Biological, Pathogen and Virus Detection (BPVD) methods and systems (or Bio Viral Sensor Detection methods and systems) are highly specialized techniques and systems for the full and complete processes of selection and training of canines for detection of viruses and/or other pathogens on humans or on surfaces. The present methods and systems also include protocols and techniques utilizing canine olfaction for the purpose of screening, searching, locating and indicating the presence of a specific biological pathogen, such as a virus, which may include, but is not limited to, COVID-19 and/or variants thereof. The present Bio Viral Sensor Detection training program, utilizes a systematic approach to training a canine, including the implementation of milestones within the program.

### BACKGROUND

Undetected pathogens may spread in public spaces, particularly if persons or objects have the pathogen thereon or carry the pathogen, but the presence of such a pathogen is not detected.

### BRIEF SUMMARY OF THE INVENTION

Provided are methods of detecting a biological pathogen, such as a virus, that include conditioning a canine to search for and passively indicate a specific odor indicating presence of the pathogen; permitting the canine trained to detect the pathogen using canine olfaction, to roam a room or other area to search for the biological pathogen; and determining from a passive reaction of the canine, whether the pathogen is present in a location or whether an individual is infected with or carrying the pathogen. In non-limiting example embodiments, the canine is conditioned to search for and passively indicate a specific odor indicating presence of a pathogen using one or more biomarkers selected from sweat, saliva and a pathogen. The sweat may be sweat from human tissue, for example from a tissue bank or from a live donor.

Also provided are methods of training a canine for biological pathogen detection. Example methods include screening a canine for detection of a biological pathogen on humans or other mammals or on surfaces, where the screening includes screening one or more canines for potential drive, character and probabilities that lead to other required performance capabilities; socializing said canine; teaching the canine basic obedience; introducing the canine to at least one biomarker selected from sweat, saliva and a pathogen, and calibrating the canine's ability to recognize, learn and maintain an ability to detect parts per trillion and lower of a pathogen.

### DETAILED DESCRIPTION OF THE INVENTION

The present invention provides viral sensor detection methods, methods of training a canine for viral sensor detection methods, and methods of screening a canine for viral sensor detection. The term “viral sensor detection” is also intended to encompass detection of other biological pathogens in addition to viruses.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language).

The present methods and systems may be useful in many settings. By way of non-limiting example embodiment, trained canines in accordance with the present invention may be used to detect biological pathogen(s) for example, at airports, train stations, bus stations, cruise ports, concert and sports venues, office buildings and other workplaces, government buildings, parks, monuments, schools, universities, nursing homes, hospitals, and numerous other places where it may be useful to be able to detect biological pathogen(s), such as a virus. If a location is detected to have the pathogen/virus present, it may then be sanitized to try to prevent further spread of the pathogen. Persons who had contact with that area may wish to be tested. If a person is detected to have a pathogen/virus, numerous possible actions may be taken. For example, the person may not be permitted admission to the venue or not allowed to board (in the case of transportation), they may be asked to leave the venue, they may be subjected to further screening (such as a rapid test), and/or taken away from others in the vicinity, to prevent transmission of the pathogen to others at the vicinity. Other immediate and non-immediate actions are contemplated for the benefit of the public as a whole in reducing transmission of the pathogen, and for the benefit of the individual in being able to obtain further testing and/or treatment. The present methods have the effect of reducing screening time for certain pathogens from days or weeks in some cases, to seconds. This may help prevent the spread of pathogens, particularly for persons who are have been tested and have not yet received results and/or when carriers of the pathogen are not symptomatic and/or have not been tested at all.

Provided herein are methods of detecting a biological pathogen, that include conditioning a canine to search for and passively indicate a specific odor indicating presence of the pathogen; permitting the canine trained to detect the pathogen using canine olfaction, to roam a room or other area to search for the pathogen; and determining from a passive reaction of the canine, whether the pathogen is present in a location or whether an individual is infected with or carrying the pathogen. In example methods, the canine is conditioned to search for and passively indicate a specific odor indicating presence of the pathogen using one or more biomarkers selected from sweat, saliva and a pathogen. The sweat may be sweat from human tissue. According to example embodiments, the human tissue is human tissue obtained from a tissue bank. Alternatively, the human tissue may be obtained form a live donor. In example embodiments, the pathogen may be a virus, such as COVID-19 and/or one or more variants thereof. In example embodiments, calibration is set for the canine's ability to recognize, learn and maintain parts per trillion and lower calibration amounts of the pathogen.

As described further below, the canine may be trained by taking the natural inactive bio marker of one or more pathogens that are sought to be detected using the present sensor methods, and captures the desired virus/marker, then imitates and replicates the specific molecular structure from its natural state and ensures that it is void of all other components. The natural inactive bio marker of the pathogen comprises sweat samples obtained from a human tissue, saliva or a neutralized virus.

### Methods of Training a Canine for Viral Sensor Detection

According to example embodiments, the canine training methodology is based on both continuous and variable positive reinforcement schedules, using the principles of instrumental learning. In non-limiting examples, every aspect of the system from initial canine selection, to every response and through all variables, temperatures, and environmental aspects, is captured, analyzed and documented with scientific rigor. In non-limiting example embodiments of the present invention, all systems of the technology from the selection and procurement of a canine, the initial imprinting and advancement to the operational application are executed with the dynamic performance.

According to example embodiments, the training methodology used to condition the canine to search for, and passively indicate a specific odor, is instrumental learning aka operant conditioning. During the odor imprinting phase of the training, the canine learns to associate the connection between:
Locating the target odor
Giving a passive indication on the odor, and
Receiving a reward for response.

In the present training methods, this chain of events is important to produce a balanced and consistent behavior repetition from the canine, and conducting this behavior/performance on a continuous basis, at such a low threshold and hold a high detection rate requires a very specific and thorough approach.

The following techniques of the present Bio Viral Sensor Detection System provide a program that is highly efficient, calibrated, and capable. Which is specifically tailored and designed for low viral levels of detection on persons or in areas of a gathering.

Non-limiting examples of the present processes are set forth below.

In particular, methods provided herein include methods of training a canine for biological pathogen detection that include screening a canine for detection of a biological pathogen on humans and/or on surfaces, in which the screening includes for example, screening a canine for potential drive, character and probabilities that lead to other required performance capabilities; socializing said canine; teaching the canine basic obedience; introducing the canine to at least one biomarker selected from sweat, saliva and a pathogen; and calibrating the canine's ability to recognize, learn and maintain an ability to detect parts per trillion and lower of a pathogen. The sweat may be sweat from human tissue, for example from a tissue bank or from a live donor. The present methods may be calibrated to adjust to any type of reward schedule, i.e., fixed ratio, fixed interval, variable ratio, and fixed ratio, or variable interval reinforcement schedules.

The present methods may further include teaching the canine to passively indicate a marker, for example, by the canine being taught to give a sit response in front of the trainer during reward play to elicit the throwing of the retrieve reward toy or delivered a treat. The obedience and commanded behaviors may be completed without cue, treat or reward. In non-limiting examples of the present invention, the biological pathogen is a virus. The virus may be, for example, COVID-19 and/or one or more variants thereof, but is not limited to this.

### Methods of Screening a Canine for Viral Sensor Detection

According to example embodiments, the present invention includes specific procurement and development systems, regarding an initial selection and placement of canines into the Bio Viral Sensor Detection System. The first step in developing an extremely high-performance biological sensor system based on the Bio Viral Sensor Detection system is a specific process for the testing, selection and procurement of canines that are suitable for viral sensor detection training and performance. The canine should be in excellent health with no acute or chronic disease or condition. A canine must display potential drive, character and probabilities that lead to other required performance capabilities. A test must be conducted that involves the cognition and physical behaviors and abilities to ensure that the afore-mentioned characteristics are maintained, while the canine also maintains the ability to demonstrate more calm, methodical and intense search activities. Initial veterinarian assessments that include unflawed blood panels and x-rays that are rated by PennHIP at good or better prior to incorporating the canine into the program.

Examples of the present Bio Viral Sensor Detection system implements protocols that during preparation and throughout its training period, the canines will be socialized, taught basic obedience and introduced to the marker (aka clicker or tone). The standard requirements are to Heel, Down, Hold position/Stay, Come, and Sit. Note: Positive reinforcement is to be used throughout all training. In non-limiting examples, thirty minutes of exercise, grooming and health checkpoints may be conducted for each canine on a twice a day basis. During the initial training periods, the canines are taught to give a sit response in front of the trainer during reward play to elicit the throwing of the retrieve reward toy or delivered a treat. All obedience and commanded behaviors are to be completed without cue, treat or reward. Protocols and procedures are utilized in this time period to familiarize and acclimate the canines to the learning environment, climate and training equipment.

In example embodiments, a specific and unique laboratory is required for canine biological pathogen sensor training. To ensure proper calibration is set for the canine's ability to recognize, learn and maintain an extremely low, such as parts per trillion and lower calibration threshold is required. This method ensures this is accomplished by implementing a unique, specific and sterile training environment in order to ensure contamination, and odor transfer is relieved from the process. In addition, the Bio Viral Sensor Detection canine training is performed in an environment where distractions, biases, and other human influence is kept to a minimum. A specific protocol for sanitation and sterilization procedures is incorporated into the environment, which includes for example, autoclave and other sterilization and/or decontamination tools that afford the highest levels of contamination prevention. Additionally, the present Bio Viral Sensor System canine training requires a work site that is a climate-controlled environment, free of distractions, which will ensure the efficacy and concentration of performance to the canine for longer periods of time. In example embodiments, specific target/filter holders, analysis stands, and other equipment made of a non-porous, medical grade steel, or other suitable material, is required to ensure contamination standards are maintained.

### Canine Viral Sensor Training

In example embodiments, throughout the canine training process, a precise data gathering system may be maintained and utilized to ensure proper calibration is set for the canine's ability to recognize, learn and maintain an extremely low calibration threshold for detecting a viral sensor. To ensure this is accomplished without error, an automated COB (change of behavior) FR (final response) reader incorporates laser technology that reads, times, marks and relays identifiable canine changes in behavior. These changes in behavior such as (sniff rate, speed, excitement, temp, heart rate, nose at source, etc.,) are consistent with the behaviors the canine presents, when it recognizes trained target material such as the virus or markers of the virus.

Examples of the present Bio Viral Sensor Detection system also require a very specific approach to the reward schedule. The above-mentioned technology (COB FR reader) ensures that the present system is applied in a set or variable manner, whichever is required depending upon the stage of training. This system can be calibrated to adjust to any type of reward schedule, i.e., fixed ratio, fixed interval, variable ratio, and fixed ratio, or variable interval reinforcement schedules. This device can also capture, critical response—mark times, and possible false to positive find rates and other data that is invaluable to maintain successful viral detection rates.

### The Specific and Unique Training Aid, Component

To confirm and validate that the target odor is captured and presented for training and in some applications, in addition to the previous-mentioned required sanitation and sterilization protocols, the Bio Viral Detection System utilizes the following products, techniques, protocols and methods: A specific and unique system is used for a safe, manageable, and efficient training target. Examples of the present system take the natural bio marker of a virus (such as COVID-19 and/or variants thereof, for example) that is sought to be detected using the present sensor methods, and captures the desired virus/marker, then imitates and replicates the specific molecular structure from its natural state, and ensures that it is void of all other components. That is, an inactive bio marker is used to avoid potentially contaminating the canines and/or any human trainers. The procedure then takes the previous described target, ensures the target is safe and non-hazardous, and then concentrates the target to an increased level. A target may be for example, the virus, or markers (effects) of the virus, anything that trainers, imprint into the canine olfactory. This provides a magnified scent picture (the picture of how the canine sees the odor/target odor with its olfactory system), an increased head space to a level that is not naturally available but more recognizable to a canine for training and imprinting purposes. Headspace technology is a technique developed in the 1980s to elucidate the odor compounds present in the air surrounding various objects. In addition, this same procedure conducted in reverse. Meaning that the threshold that occurs naturally, can be reduced, and lowered which affords this system to increase the viral detection rates to maximum potential and provide critical data for testing capabilities. In these embodiments, a canine may be trained to be able to detect very low levels of a virus.

In example embodiments, data may be recorded that is associated with canine, handier, environment, time, other schedules, training, results of training, climate, and genetics. This information may be used for example to calibrate and maintain performance of canines at low levels of pathogen detection; wherein said matrix captures and maintains data that is associated with canine, handler, environment, time, schedules, training, results of training, climate, and genetic information of the canine. The information may also be used to predict success.

To ensure the present Bio Viral Sensor Detection system is providing a standard and maintaining a high degree of viral detection in different applications, according to example embodiments, three targets are implemented into the present training program. 1) Sweat: the bio marker may be removed from tissue of a human subject who has tested positive for the virus, and is provided as described herein, the tissue may be from a live subject or obtained from a tissue bank. 2) Saliva: the bio marker is obtained from a human subject who has tested positive for the virus and is provided to the canine as described above in the Training Aid section. 3) Virus: a sample of the virus is obtained and “neutralized” or “suspended” in a manner that is non communicable, safe, and manageable. In the sample of sweat, locate and remove the bio markers. Remove the bio markers (VOCs) that indicate a virus is present in the individual. That bio marker, will be placed in a training aid. According to further example embodiments, multiple sweat and/or saliva biomarkers may be used.

The biomarkers may be transported using e.g., deep cold storage transport, such as that presently being used by FEDEX to keep vaccines, samples, and other medical items cold, such as 70 degrees below zero (F) or colder. The present invention is not limited to use of a particular carrier, as long as the transport system can maintain the temperature at or below 70 degrees below zero (F).

According to example embodiments, to ensure the Bio Viral Sensor Detection system is providing a standard to the highest degree of application, the saliva category is developed with the concept of a unique and specially designed disposable instrument/capture device (a breathalyzer type/straw), that includes a filter. To obtain the saliva specimen for training, an individual blows, coughs, spits, into the straw, a filter captures the specimen, the filter is then placed under UV light ensuring neutralization of any communicable disease or virus. Then, the filter is placed into a stand or bracket. The bio sensor (canine) analyzes the provided filters, if the biological sensor alerts or indicates, the filter is considered to contain the virus or markers of the virus. The canine trained final response may be a focused sit, for example, such as the dog sitting, nose, head and body positioned on or close to source of odor.

According to example embodiments, to ensure the Bio Viral Sensor Detection system is providing a high degree of application, the sweat category is developed with the concept of the presentation of a canine to a screen, and searching individuals in lines, or a specific search pad where the canine has for example, a 360 degree access to a person. In these methods, an individual stands in an identified area and opportunity is provided to present the individual to the canine for sniff and analyzing/detecting of a viral target. The bio sensor (canine) analyzes the individual, and the individual's clothing, and if the biological sensor alerts or indicates, individual is considered to have the virus or markers of the virus.

According to example embodiments, to ensure the Bio Viral Sensor Detection system is providing a standard to the highest degree of application regarding the search, clearing and detection of the biological pathogen, such as a virus, itself, the virus category is developed. A Bio Viral Sensor Detection canine is presented to a searchable room/area. The room or area does not need to have a human present (with sweat or saliva). This specific canine may be trained in a manner where the productive areas are different than a conventional detection canine approach. For example, the canine may be trained to detect a pathogen, such as a virus, on non-human surfaces, such as on doorknobs, counters, desks, water fountains, food, etc. In example methods, the Bio sensor (canine) is permitted to search and/or analyze the area or room. If the biological sensor alerts or indicates, then the area or location is considered to contain the virus.

The present Bio Viral Sensor Detection training system, is a system that allows a canine to be trained to detect a single pathogen, such as a virus such as COVID-19 and/or variants thereof. In example embodiments, the detection of other markers and viruses are viable to be placed into the canine's repertoire of learned targets.

A milestone progression system is utilized to ensure that all canines in the Bio Viral Sensor Detection system achieve a specific rate of learning, progression and success. This is an important component at thresholds of such a low magnitude.

Initial training after the period of acclimation and familiarization includes utilizing a detection odor calibration that is not located, found nor exist in any environment. This odor will be the initial odor in the canine's repertoire of trained odors that will be utilized to teach first steps of the process, that include eliciting a change of behavior that indicates a trained odor is present.

This stage introduces the target odor to the canine using either the Filter stands, Scent Introduction Box (SIB) or the Scent Carousel.

In non-limiting example embodiments of training a canine using the present methods, at no stage is the handler to have any interaction with the canine during the search, the canine must learn to search independently and must learn itself that the correct response to odor is the ‘SIT’ position (or other indication that may be decided by the trainer). The canine may show different behaviors prior to adopting the correct one; this is a normal consequence of the imprinting process. The canine must only receive its reward for carrying out the correct responses; no interaction is given for the incorrect behaviors. Only when the reward has been delivered by the trainer is there to be interaction with the canine, giving it both verbal and physical praise, and delivering the reward for a play (reward) stage.

Other example Bio Viral Sensor Detection Training system principles within the scope of the present invention, may include the following according to non-limiting example embodiments.

In example embodiments, the canine will be required to pass specific and detailed criteria, milestones, benchmarks and progress checks that are within the training plan.

In example embodiments, the training plan calls for a variable reward schedule will be implemented after a set point or specific stage and should be used throughout the following stage and continued for the remainder of training.

In example embodiments, initially blank target mediums will be utilized prior to actual target being presented in order to teach the canine that the filter or medium is not a target.

In example embodiments, all mediums and/or filters are always to be handled according to Bio Viral Sensor Detection protocols and in compliance with Federal standards.

Once the canine can search all the stands, platforms, or presentations, and is reliably indicating multiple amounts of the target odors, at a 52% success rate for example, distracters can be added to a portion of random mediums. The canine must be reliably indicating on the target medium before distraction filters or medium are introduced.

Now the target material can be introduced, initially a concentrated magnified level is used but it is critical that the amount of molecules needs reduced quickly.

The canines will have different scenting abilities and it is important to maintain and manage accurate records to monitor the training progress and ensure each canine receives the level of training appropriate to their abilities.

In example embodiments, after a canine is introduced to stands for the filters, the stands and number of filters may be increased for analyzation. Initially one holder/stand may be placed in the training room containing a target filter. The canine is prepared as previously in imprinting sessions.

At no point should the trainer present the holder/filter/medium to the canine, all search and performance for the target will be conducted off leash without handler steering without handler or trainer shaping.

Example systems implement a screen/blind where the view is one way, where the handler/trainer has visual of the canine activity, but the canine has no visual of handler/trainer/observer, in order to prevent influence.

The following repetitions will increase the quantity of stands, with each newly introduced stand containing a blank filter. Initially, the approach is with target on second filter, for canine searches the first holder as a blank before hitting target. First medium or filter will not always be void of target. The system is repeated with the target filter being moved to different locations within the line of holders.

The canine must then complete successfully complete all prior criteria with a 60% success rate (for example) before progressing to a variety of filters with distraction odors and lines of holders with no target. Variable reward should be used throughout this stage.

Once all criteria is complete, a double blind test will be conducted, with at least a 93% success rate required to validate that the canine has made a successful criteria on the target/component/material. It is contemplated that other success rates, such as at least 92% may be suitable for validation.

The canine will then proceed into a operational readiness phase, where the imprinted canine is moved into an acclimation familiarization training phase. This phase includes placing the canine in an associated environment for comfortability and soundness for a short period of time, until the canine displays a workable readiness. At this time the trained target will be utilized in these areas in the specified training mode. Filter, Clothing, Virus. After the canine displays confidence and detection rates are associated with initial sterile training, a double-blind test.

After all training criteria is complete, a double blind test will be conducted, with at least a 93% success rate required to validate that the canine is validated as a Bio Viral Sensor Detection Canine and is operationally ready. It is contemplated that other success rates, such as at least 92% may be suitable for validation.

While the present disclosure has been described in terms of exemplary aspects, those skilled in the art will recognize that the present disclosure can be practiced with modifications in the spirit and scope of the application. For example, while the present description refers to detection of a virus (such as COVID-19 and variants thereof) in particular, it is contemplated that other pathogens may be detected using the present methods and systems, and the application should be considered to include such pathogens. These examples given above are merely illustrative and are not meant to be an exhaustive list of all possible designs, aspects, applications or modifications of the present disclosure.

We claim:

1. A method of detecting a biological pathogen, comprising
conditioning a canine to search for and passively indicate a specific odor indicating presence of the biological pathogen, wherein said canine is conditioned to search for and passively indicate a specific odor indicating presence of the pathogen using one or more biomarkers selected from the group consisting of sweat, saliva and a pathogen;
permitting the canine trained to detect the pathogen using canine olfaction, to roam a room or other area or be exposed to a human to search for and detect the pathogen; and
determining from a passive reaction of the canine, whether the pathogen is present in a location or whether an individual is infected with or carrying the pathogen.

2. The method of claim 1, wherein the pathogen a virus.

3. The method of claim 2, wherein the virus is COVID-19 or a variant thereof.

4. The method of claim 1, wherein the sweat is sweat from human tissue.

5. The method of claim 4, wherein human tissue is from a tissue bank

6. The method of claim 4, wherein human tissue is from a live donor.

7. The method of claim 1, wherein calibration is set for the canine's ability to recognize, learn and maintain parts per trillion and lower calibration amounts of the pathogen.

8. The method of claim 1, wherein the canine is trained by taking the natural inactive bio marker of a pathogen that is sought to be detected using the present sensor methods, and captures the desired virus/marker, then imitates and replicates the specific molecular structure from its natural state and ensures that it is void of all other components.

9. A method of training a canine for biological pathogen detection comprising
screening a canine for biological pathogen detection, wherein said screening includes screening a canine for potential drive, character and probabilities that lead to suitable performance capabilities;
socializing said canine;
teaching the canine basic obedience;
introducing the canine to at least one biomarker selected from the group consisting of sweat, saliva and a pathogen; and
calibrating the canine's ability to recognize, learn and maintain an ability to detect parts per trillion and lower of a pathogen.

10. The method of claim 9, wherein the method is calibrated to adjust to any type of reward schedule, including at least one reward schedule selected from the group consisting of fixed ratio, fixed interval, variable ratio, and fixed ratio, or variable interval reinforcement schedules.

11. The method of claim 9, further comprising teaching the canine to passively indicate a marker, by the canines being taught to give a sit response in front of the trainer during reward play to elicit the throwing of the retrieve reward toy or delivered a treat;
wherein obedience and commanded behaviors are to be completed without cue, treat or reward.

12. The method of claim 9, wherein said biological pathogen is a virus.

13. The method of claim 12, wherein the virus is COVID-19 or a variant thereof.

14. The method of claim 9, wherein the sweat is sweat from human tissue.

15. The method of claim 14, wherein human tissue is from a tissue bank

16. The method of claim 14, wherein human tissue is from a live donor.
